(19) United States
(12) Patent Application Publication
(10) Pub. No.: US 2004/0136917 A1
(43) Pub. Date: Jul. 15, 2004

(54) Methods of stimulating immune response in virally infected individuals

(76) Inventor: Eugene Mandrea, Palos Heights, IL (US)

Correspondence Address: BARNES & THORNBURG, P.O. BOX 2786, CHICAGO, IL, 60690-2786, US

(21) Appl. No.: 10/751,371

(22) Filed: Jan. 5, 2004

Related U.S. Application Data
(60) Provisional application No. 60/438,431, filed on Jan. 6, 2003.

(51) Int. Cl. A61L 9/04; A61K 31/4745

(52) U.S. Cl. 424/45; 514/292

(57) ABSTRACT

Methods to stimulate host immune system against viral infections associated with common colds are disclosed. Methods to stimulate immune response of a virally infected individual through an immuno modifier such as a non-nucleoside imidazoquinolinamine (heterocyclic amine) are disclosed.

### CROSS-REFERENCE TO OTHER APPLICATIONS

This application claims priority to U.S. Ser. No. 60/438,431 filed Jan. 6, 2003.

### FIELD

Methods to stimulate host immune system against viral infections associated with common colds are disclosed. Methods to stimulate immune response of a virally infected individual through an immunomodifier such as a non-nucleoside imidazoquinolinamine (heterocyclic amine) are disclosed.

### BACKGROUND

Uncomplicated cases of viral infections usually produce mild symptoms such as nasal discharge, obstruction of nasal breathing, swelling of the sinus membranes, sneezing, sore throat, cough, and headache. These symptoms generally last between one and two weeks. A mild infection is generally associated with the rhinoviruses and the coronaviruses. The uncomplicated infection is most often referred to as the “common cold”.

At present, only symptomatic treatment is available for uncomplicated viral infections, “common colds”. The treatments include the use of over-the-counter decongestants, cough suppressants, cough expectorants, aspirin, and acetaminophen. The treatments, however, do not cure or even shorten the duration of the illness. Moreover, many of the treatments have side effects such as drowsiness, dizziness, insomnia, or upset stomach. Because of the diversity of the viruses, vaccines may not be effective in preventing the onset of colds.

It has been estimated that in the course of a year individuals in the United States suffer one billion colds. Colds thus have a tremendous societal cost in lost work days and lost school days. People suffer symptomatic discomfort. Even people receiving symptomatic treatment still suffer from some discomfort and additionally suffer side effects of treatment.

Aldara™ (imiquimod; manufactured by 3M corporation, St. Paul, Minn.) cream, is a prescribed patient-applied topical cream for treating external genital and perianal warts. Aldara™ product label does not recommend using it for any other purposes.

### SUMMARY

Methods to reduce the duration of symptoms associated with the common cold or viral rhinitis, without producing any substantial side effects generally associated with symptomatic treatment are disclosed. To reduce the duration of symptoms associated with the common cold, methods relate to applying an imidazoquinolinamine formulation, such as, for example, an imiquimod salve within a person's nostrils, also referred to as nares. Any suitable imidazoquinolinamine formulation can be used to reduce the duration of symptoms associated with the common cold or viral rhinitis.

Application of imiquimod to the inside of the nostrils and in particular to the mucosal membrane of an infected individual stimulates host cells to secrete chemical substances such as interleukins and interferons that promote the individual's immune response.

A method to reduce the duration of symptoms associated with the common cold or viral rhinitis includes application of ½ packet of Aldara™ (imiquimod formulation; 0.25 g of 5% active ingredient) into both nostrils (nares) every 12 hours for a total of 4 applications. The formulation may be applied by way of an applicator or any other suitable means. The formulation is applied into both nares at the onset of the cold. The onset is the day when the first cold symptoms appear. If the formulation is not applied on the first day the symptoms appear, it should be applied by the next day. The formulation is applied twice daily for two consecutive days. The formulation can be massaged into the internal surface of each nare. The treatment of the second nare is after the treatment of the person's first nare.

An imiquimod formulation is applied as described above at the onset of first cold symptoms such as nasal irritation, watery eyes, nasal drip or other early cold symptoms. The earlier the imiquimod formulation is applied after the onset of the cold, the shorter the recovery from cold. An imiquimod formulation may also be applied the next day after the onset of cold.

A method to reduce the duration of symptoms associated with the common cold or viral rhinitis includes application of a coating of the mucosal membrane within each nare with Neosynepherine, prior to applying the Aldara™ formulation within each nare The Neosynepherine may be applied in the form of an over-the-counter liquid formulation by means of a spray bottle. The Neosynepherine is preferably applied 15 minutes before applying the imiquimod formulation.

Other novel features, characteristics and aspects of the methods described herein can be further understood with reference to the below described drawings, detailed description, examples, and the appended claims.

### BRIEF DESCRIPTION OF DRAWINGS

The drawings are provided to illustrate some of the embodiments of the disclosure. It is envisioned that alternate configurations of the embodiments of the present disclosure maybe adopted without deviating from the disclosure as illustrated in these drawings.

FIG. 1 pictorially illustrates how cytokines promote and regulate the immune cell response;

FIG. 2 pictorially illustrates further how cytokines help to regulate and promote the body's immune response;

FIG. 3a shows a side and top perspective view of a swab-type applicator for use with an imiquimod formulation.

FIG. 3b shows a side and top perspective view of a spray nozzle coupled to a bottle; the spray nozzle is the applicator for a liquid imiquimod formulation.

FIG. 3c shows a cross-sectional side view of an injection tube interfaced with a hollow swab head which can be used to apply an imiquimod formulation; the injection tube is connected to a vessel and the vessel has a piston actuator to inject a certain amount of imiquimod through the swab head into a nare.

FIG. 3d shows a side and perspective view of a dropper-type nozzle which dispenses liquid imiquimod in droplet form; the nozzle is connected to a squeeze bulb.

FIG. 4 pictorially illustrates a possible mode of action of an imidazoquinolinamine such as imiquimod in stimulating host immune system.

FIG. 5 shows the structural formulae for imiquimod and resiquimod.

### DETAILED DESCRIPTION

While the concepts of the present disclosure are illustrated and described in detail in the drawings and the description below, such an illustration and description is to be considered as exemplary and not restrictive in character, it being understood that only the illustrative embodiment is shown and described and that all changes and modifications that come within the spirit of the disclosure are desired to be protected.

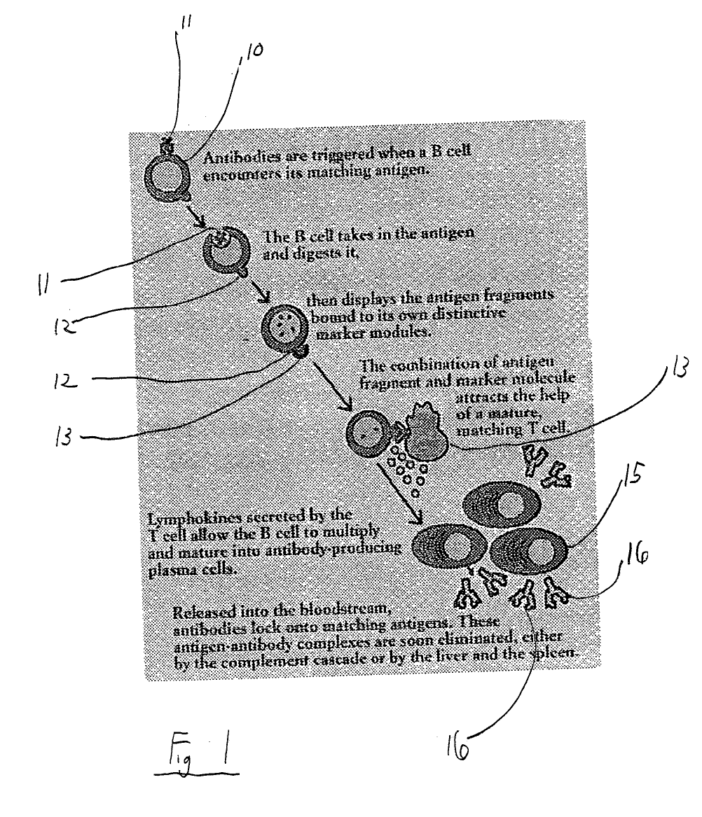

FIG. 1 generally describes how some cells in the human body operate as part of the host immune system to combat infection. In FIG. 1, a lymphocyte (monocytic dendritic cells) 10 takes in an antigen 11 and displays part of the digested antigen 13 with a marker molecule 12 to a mature T cell 13. The T cell secretes cytokines 14 which help stimulate the B cell to mature into a plasma cell 15 which produces antibodies 16. The foreign antigen in the present diagram is viral. This is known as T-helper 2 mode.

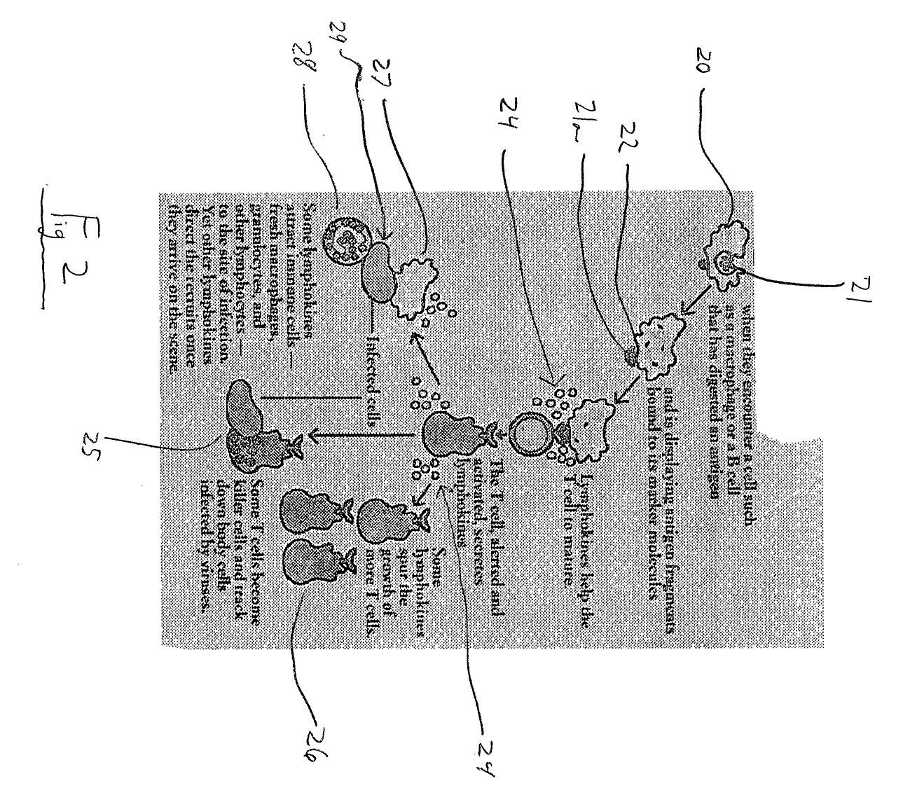

This figure is a shematic representation of the aquired immune system which works much more slowly then the innate immune system. As part of the innate immune system, the skin and mucus membranes have been showed to be able to produce and secrete cytokines such as TNFa, and the like. FIG. 2 discloses a macrophage 20 digesting a foreign antigen 21. The macrophage 20 displays antigen fragments 21a on its marker 22 to an immature T cell 23. Cytokines 24 are produced and help the T cell mature. Further cytokines 24 actually produced by the maturing T cell help the maturing T cell evolve into killer cells 25 and helper T cells 26. Cytokines 24 also help attract additional macrophages 27, granulocytes 28, and other lymphocytes to the area of infection thereby promoting an attack on infected cells 29 (this is now known as T-helper 1 mode).

Imiquimod enhances both the innate and cell-mediated immune pathways to stimulate the production of various cytokines. For example, imiquimod stimulates the innate immune response by inducing the synthesis and release of cytokines, including IFN-α and TNF-α in both humans and animal studies. Production of various cytokines by the activated innate immune system results in the strengthening of the cell-cell interaction. For example, monocytes, macrophages, B cells, and dendritic cells (including Langerhan cells; LC) are targeted by imiquimod.

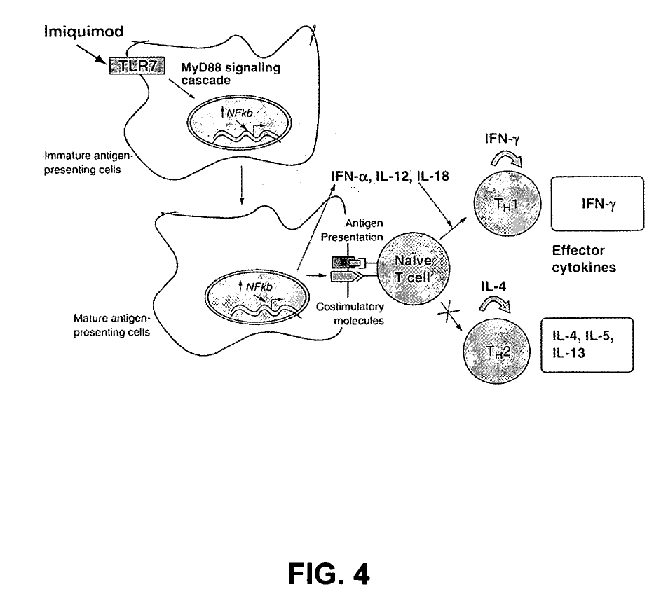

A proposed mechanism by which imiquimod may activate the above-mentioned target cells is via the activation of Toll-like receptors (TLRs), a family of pathogen recognition receptors located on the cell surface of various innate immune cells such as dendritic cells. Activation of TLRs, such as, for example, TLR7 results in the downstream activation of a signal cascade mediated by Myd88 and various effector cytokines such as IFN-α, IL-12, and IL-18 are produced (FIG. 4).

A proposed mechanism of action for imiquimod to activate the cell-mediated immune response is through an indirect stimulation of T-cells by producing Th-1 cytokine IFN-γ. Imiquimod also enhances the migration of LCs to the regional lymph nodes to enhance antigen presentation to T-cells. In vitro assays have established that exposure of LCs to imiquimod results in increased gene expression for TF-α, IL-1β, and IL-12, and also secretion of IFN-γ by imiquimod treated T-cells compared to untreated cells.

Studies have shown that immune response modifiers such as imiquimod and resiquimod are TLR7 agonists and induce type 1 interferon in numerous species including humans. Imiquimod and resiquimod induce IFN-α and IFN-Ω from purified plasmacytoid dendritic cells.

Thus imiquimod and resiquimod stimulate the local production of various cytokines such as IL-12, IL-18, IL-1β, IFN-α, and IFN-γ to promote both innate as well as cell-mediated immunity.

The common cold causes a group of symptoms that usually are easily recognized by patients and doctors. About 50 percent of patients will develop a sore throat, which is often the first symptom to appear since it can occur as early as 10 hours after infection. This is followed rapidly by the most common symptoms of the common cold—congested nasal and sinus passages, a runny nose and sneezing. Hoarseness and cough are less likely to occur, but they may last longer than other symptoms, sometimes for several weeks.

Most patients diagnose the common cold by the typical symptoms of runny nose, congestion and sneezing, and rarely consult medical attention. Symptoms typically peak on the second, third or fourth days of infection and last about one week to 10 days. Up to 25 percent of people may have persistent symptoms, such as a nagging cough that can last for several weeks.

The methods disclosed herein stimulate the immune system response as described in FIGS. 1, 2, and 4. The methods disclosed herein promote host cells to secrete chemicals and cytokines such as interferons and interleukins, which impact the host cellular immune response at least partially as shown in FIGS. 1, 2, and 4.

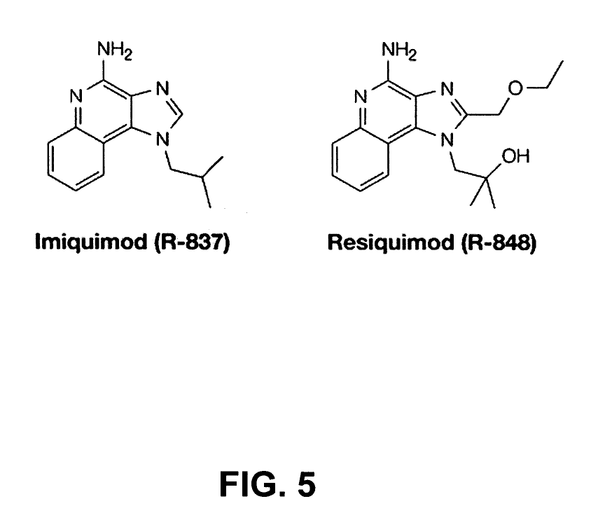

Low molecular weight heterocyclic non-nucleoside imidazoquinolinamines can be used to treat viral rhinitis. One such imidazoquinolinamine is imiquimod whose IUPAC nomenclature is (1-(2-methylpropyl)-1H-imidazo)[4,5-c] quinolin-2-amine. Imiquimod may also be referred to as R-837. Another imidazoquinolinamine is resiquimod, whose IUPAC nomenclature is 4-amino-2-ethoxymethyl-α,α-dimethyl-1H-imidazol [4,5-c] quinoline-1-ethanol. Resiquimod may also be referred to as R-848. (see FIG. 5).

To provide effective treatment, a formulation of imiquimod commonly used to treat warts can be used. For example, the formulation sold in salve format under the brand name Aldara™ is effective. It is believed that other imiquimod formulations such as imiquimod in a fluid formulation or in a fine powder formulation might be effective.

A method to reduce the duration of symptoms associated with the common cold or viral rhinitis includes application of ½ packet of Aldara™ (imiquimod formulation; 0.25 g of 5% active ingredient) into both nostrils (nares) every 12 hours for a total of 4 applications. Each gram of 5% Aldara™ cream contains 50 mg of imiquimod as active ingredient.

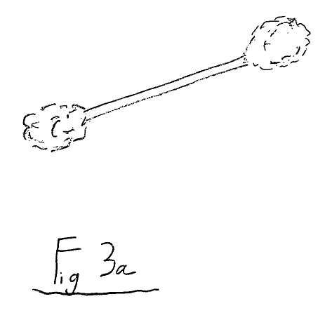

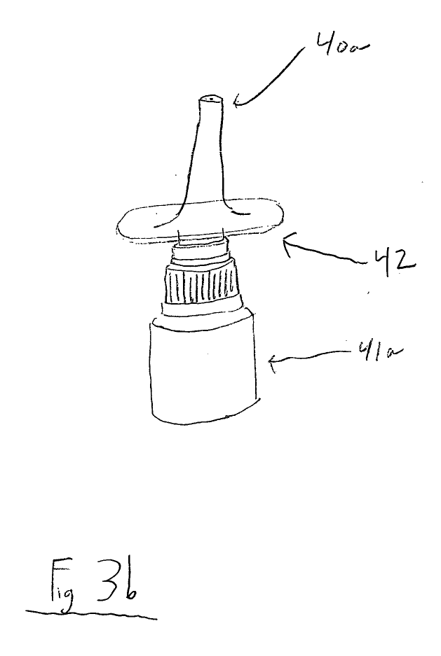

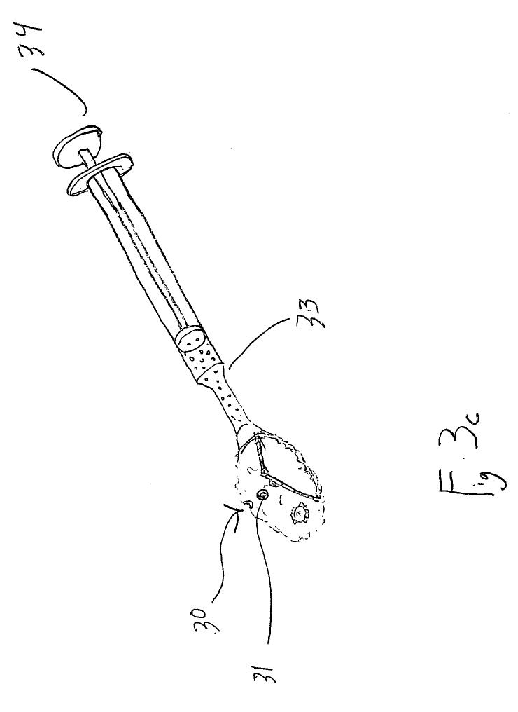

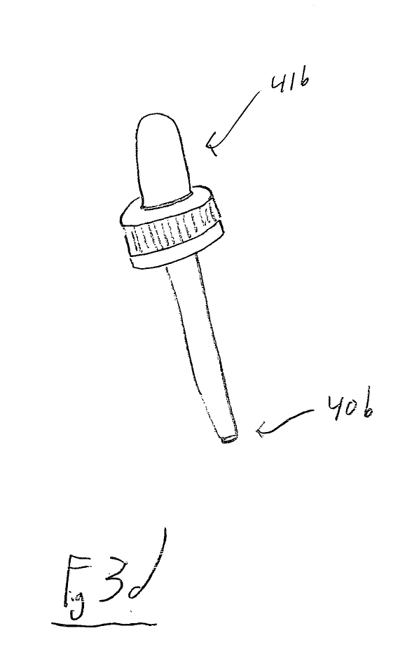

In an embodiment, an applicator is used to disperse the imiquimod within each nare. The applicator used can be a cotton swab. See FIG. 3a. The swab should be of suitable size to fit internally within each nare such that the exterior of the swab can move freely within each nare and make substantial contact with the nares mucosal membrane. In an embodiment, the imiquimod salve is combined with the swab shown in FIG. 3a by applying a 4 mm³ dab of the imiquimod salve on the head of the swab. The swab is then inserted in a nare and moved around within the nare so as to spread the salve over the nare's mucosal membrane.

The swab shown in FIG. 3a is not the only type of applicator which can be used to apply an amount of imiquimod salve to a nare. Many other types of applicators can be used. For instance, an applicator with a hollow swab head fluidly connected with an injection tube would work. See FIG. 3c. The hollow swab head 30 would have a series of tiny apertures 31 through which the salve could be extruded. Extrusion through the tiny holes would occur by way of actuating an amount of salve in the injection tube 33 to flow into the hollow swab head 30. Actuation could occur by a plunger 34. Once the salve is extruded, the hollow swab head is moved around within the nare so as to spread the salve within the nare. The swab head could have many configurations. Additionally, it is feasible that one could use an injection tube alone to dispense the salve in the nare. The salve could then be spread by a cotton swab, a finger or some other means.

Instead of using imiquimod in a salve formulation, one may also use imiquimod in a liquid formulation. If an imiquimod liquid formulation is used, the applicator can be a spray nozzle 40a, FIG. 3b, or a dropper nozzle 40b, FIG. 3d. The nozzles 40a, 40b could be interfaced to vessels such as bottles 41a or squeeze bulbs 41b. The vessels 41a, 41b and nozzles 40a and 40b would be configured so that a predetermined actuation sequence would emit an effective dosage of imiquimod from the nozzle into the nare. For instance, to emit imiquimod from the spray nozzle 40a, an operator would simply depress pump 42 interfaced with the spray nozzle. In addition to the above, the applicator could simply be a finger or any other member which would fit within a nare and allow dispersion of the imiquimod formulation within the nare.

It is preferable after first applying the imiquimod formulation to the internal surface of a nare or nares, i.e., within the nare, to massage the formulation into the nare's mucosal membrane.

Each of a person's two nares is treated in the same fashion. Treatment of the second nare is immediately after the first nare. Massaging of the salve into at least a portion of the internal surface of each nare can occur after the salve has been applied to both nares.

Prior to treating each nare with the imiquimod formulation, each nare can be precoated with Neosynepherine (a solution of about 10% phenylephrine hydrochloride) mat least 15 minutes before applying the imiquimod formulation. Prepping the nares with the formulation facilitates prolonged contact of the imiquimod to the nares' internal surfaces by helping to prevent wash-off due to nasal secretion. Phenylephrine is a decongestant that works by constricting (shrinking) blood vessels (veins and arteries). Constriction of blood vessels in the sinuses, nose, and chest allows drainage of these areas, which decreases congestion. Any other suitable alpha-adrenergics or other decongestants may also be used.

The utility of the above-described method for treating persons with viral infections can also be seen by reference to the below to in vivo experiments.

In each of the tests an imiquimod salve was used. The formulation was that commonly used to treat warts and sold under the brand name Aldara™. The salve was applied to each nare of the test subject by use of a common cotton swab. Either a 4 mm³ dab of salve or ½ pack of 5% Aldara™ was placed on the swab head. The swab head was inserted into a nare. The swab was moved around inside the nare to distribute the salve over the mucosal membrane of the nare. Immediately, after application of the Aldara™ to the subject's first nare, a swab was used to apply the Aldara™ to the subject's second nare. Immediately after application to each nare, the salve was massaged into the mucosal membrane of each nare.

Application of an immunomodifier such as imiquimod to the internal surface of the nostrils stimulates innate immunity locally and thus helps to shorten the duration of cold symptoms.

### EXAMPLES

Example 1: Alleviation of Viral Rhinitis Symptoms by Administering Imiquimod at or about the Onset of the Cold Symptoms

A test sample of six patients was treated for viral rhinitis using imiquimod (5% topical cream Aldara™, manufactured by 3M corporation, St. Paul, Minn.). The patients were diagnosed with viral rhinitis due to initial symptoms such as congested nasal passages (rhinitis), nasal drip or rhinorrhea, and sneezing. Contents from one half packet of a standard imiquimod formulation such as, for example, Aldara™ were applied with a cotton swab into both nares by massaging gently but thoroughly. Approximately the contents from ½ packet of 5% Aldara™ was applied thoroughly along the inside surface of both nares. Initial application of Aldara™ to all the six patients occurred within 24 hours after the appearance of first symptoms resembling viral rhinitis, in order to maximize the efficiency of imiquimod in stimulating the immune system when the viral load is presumably smaller. The procedure was repeated every 12 hours for up to 48 hours. The imiquimod packets were refrigerated after opening and the remaining contents were used for subsequent applications.

The patients were monitored for changes in the viral rhinitis symptoms. No untoward side effects was reported by any of the patients through out course of the treatment. First sign of relief (reduced nasal congestion, nasal drip) was obtained between 12 and 36 hours after beginning the imiquimod treatment. Complete disappearance of symptoms (nasal congestion, sore throat, headache; malaise) was obtained within 48 hours of treatment. One patient with Ulcerative Colitis (a possible Th2 type disorder) did not suffer any further aggravation during the treatment. In an unrelated incident, not during the course of treatment, one patient inhaled imiquimod and developed severe flu-like symptoms that spontaneously subsided within 24 hours. Therefore, care should be taken not to inhale the imiquimod formulation during application in the nostrils.

The results demonstrate that an imiquimod formulation is effective in reducing the duration of symptoms during viral rhinitis or common cold (TABLE 1). The imiquimod and other related compounds such as resiquimod stimulate the immune cells both locally and also systemically to mount a defense response against the viruses. The cold symptoms subsided within 48 hours compared to about a week or 10 days for untreated viral rhinitis. Formulations of imidazoquinolinamines such as, for example, imiquimod or resiquimod can thus be effectively used to mitigate symptoms during viral rhinitis. Depending on the intensity of the viral rhinitis, an imiquimod or resiquimod formulation or the treatment plan can be modified. For example, instead of every 12 hours, the imiquimod formulation can be applied every 8 hours. In addition, appropriate modifications of the amount of imiquimod can also be undertaken. Furthermore, any suitable method of administration can be implemented, such as, for example, using a swab, or a drip applicator or as a nasal spray.

Example 2: Alleviation of Viral Rhinitis Symptoms by Administering Imiquimod after the Onset of the Cold Symptoms

Infected Test Subject #1 developed sore throat with tingling in Larynx, Pharynx and Uvula. Twelve hours later, subject 1 also developed congestion of nose. One day later, the subject's initial symptoms intensified and subject further developed systemic symptoms such as malaise and headache. More than twenty four hours after the initial viral rhinitis symptoms appeared, Aldara™ was applied in each nostril with a Q-tip swab.

A 4 mm³ dab of salve was placed on the swab head. The swab head was inserted into a nare. The swab was moved around in the nare so as to distribute the salve over the mucosal membrane of the nare. Immediately, after application of the Aldara™ to the subject's first nare, a swab was used to apply the Aldara™ to the subject's second nare. Immediately after application to each nare, the salve was massaged into the mucosal membrane of each nare. The subject was also treated with 2 teaspoons of standard cough suppressant Guaifenesin/Dextromethorphan (Wal Tussin). The next day, malaise and headache were more pronounced and Aldara™ was reapplied with Q-tip to each nostril as described above. Also, 2 teaspoons of Guaifenesin/Dextromethorphan was administered.

Two days after the first application of Aldara™, marked improvement of symptoms, including malaise and headache was observed. Three days after the first application of Aldara™, some cough and rhinorrhea persisted and by 4-5 days all of the cold symptoms subsided.

Infected Test Subject #2 developed common cold with rhinorrhea and nose congestion. Aldara™ was applied once to each nostril every day for 3 days. By day three, except for post nasal drip, other cold symptoms subsided.

It was noted the effectiveness of the treatment decreased markedly if the imiquimod formulation was applied more than 2 days after the onset of the cold symptoms. Compared to Example 1, the Aldara™ treatment described in Example 2 required longer duration for complete symptom relief (see TABLES 1 and 2). This may be due to factors such as (i) delayed application of Aldara™ after the onset of the first cold symptoms, and (ii) infrequent application (once a day compared to twice a day in Example 1). Therefore, immediate application of Aldara™ or any other imiquimod formulation after the onset of the cold symptoms may result in quicker relief of cold symptoms.

TABLE 1
Viral rhinitis and treatment data with imiquimod

| Patient | Symptoms | Day 0^{a} | Day 1 | Day 2 |
|---|---|---|---|---|
| Patients 1-6 | Local | Present | Substantial | Complete |
| | Symptoms: | | reduction | reduction |
| | sore throat; | | | |
| | nasal | | | |
| | congestion; | | | |
| | rhinitis; | | | |
| | rhinorrhea | | | |
| | Systemic | Present | Substantial | Complete |
| | Symptoms: | | reduction | reduction |
| | headache; | | | |
| | cough; | | | |
| | malaise | | | |

^{a}onset of first cold symptoms and first administration of Aldara ™. Thereafter, Aldara ™ was administered every 12 hours for 48 hours.

TABLE 2
Viral rhinitis and treatment data with imiquimod delayed administration
of imiquimod after the first onset of cold symptoms.

| Patient | Symptoms | Day 0^{a} | Day 1^{b} | Day 2 | Day 3 | Day 4 |
|---|---|---|---|---|---|---|
| Test | Local | Present | Present | Substantial | Only mild | Complete |
| Subject 1^{c} | Symptoms: | | | reduction | rhinorrhea | reduction |
| | sore throat; | | | | | |
| | nasal | | | | | |
| | congestion; | | | | | |
| | rhinitis; | | | | | |
| | rhinorrhea | | | | | |
| | Systemic | Present | Present | Slight | Substantial | Complete |
| | Symptoms: | | | reduction | reduction | reduction |
| | headache; | | | | | |
| | cough; | | | | | |
| | malaise | | | | | |
| Test | Local | Present | Substantial | Only mild | Complete | |
| Subject 2^{d} | Symptoms: | | reduction | rhinorrhea | reduction | |
| | sore throat; | | | | | |
| | nasal | | | | | |
| | congestion; | | | | | |
| | rhinitis; | | | | | |
| | rhinorrhea | | | | | |
| | Systemic | Present | Slight | Substantial | Complete | |
| | Symptoms: | | reduction | reduction | reduction | |
| | headache; | | | | | |
| | cough; | | | | | |
| | malaise | | | | | |

^{a}onset of first cold symptoms.
^{b}first administration of Aldara ™. Thereafter, Aldara ™ was administered every 24 hours up to 3 days.
^{c}cough suppressant was also administered.
^{d}Aldara ™ was administered on Day 0, when the cold symptoms first appeared. Thereafter, Aldara ™ was administered every 24 hours up to 3 days.

I claim:

1. A method of stimulating an immune response in a virally infected individual, the method comprising:
providing an imidazoquinolinamine formulation;
disposing an amount of the imidazoquinolinamine formulation into a first nare of a virally infected individual; and
covering at least a portion of the internal surface of the individual's first nare with a portion of the amount of the imidazoquinolinamine in said nare.

2. The method of claim 1, wherein the imidazoquinolinamine formulation's active ingredient is selected from the group consisting of imiquimod and resiquimod.

3. The method of claim 1, wherein the imidazoquinolinamine formulation is disposed using a device selected from the group consisting of a swab, syringe, spray nozzle, and a drip applicator.

4. The method of claim 1, wherein the imidazoquinolinamine formulation comprises imiquimod as an active ingredient in the amount of about 12 mg.

5. The method of claim 1, wherein the imidazoquinolinamine formulation comprises resiquimod as an active ingredient in the amount of about 12 mg.

6. The method of claim 1, wherein the imidazoquinolinamine formulation is selected from a group consisting of a cream, gel, liquid, paste, aerosol, and an emulsion.

7. The method of claim 1, wherein about 15 minutes prior to disposing the imidazoquinolinamine formulation, the first nare is treated with a decongestant.

8. The method of claim 7, wherein the decongestant is Neosynepherine.

9. The method of claim 1, wherein the individual is infected with viral rhinitis.

10. The method of claim 1 further comprising providing the imidazoquinolinamine formulation every twelve hours until relief from cold symptoms is obtained.

11. The method of claim 1, wherein the imidazoquinolinamine formulation is applied within twenty four hours after an appearance of first cold symptoms.
